(19) United States
(12) Patent Application Publication
(10) Pub. No.: US 2016/0106831 A1
(43) Pub. Date: Apr. 21, 2016

(54) BEVERAGE COMPOSITIONS AND OTHER FORMULATIONS FOR TREATING ALLERGIES AND METHODS FOR MAKING THE SAME

(71) Applicant: AllergyIntellect, Glenwood Springs, CO (US)

(72) Inventors: Robert McDermott, Glenwood Springs, CO (US); Neil Jain, Glenwood Springs, CO (US)

(21) Appl. No.: 14/931,364

(22) Filed: Nov. 3, 2015

Related U.S. Application Data
(63) Continuation of application No. 14/183,444, filed on Feb. 18, 2014, now abandoned.
(60) Provisional application No. 61/766,651, filed on Feb. 19, 2013.

(51) Int. Cl. A61K 39/36; A61K 36/258; A61K 31/375; A61K 31/59; A23L 2/52; A61K 9/00; A61K 35/644; A23L 2/02; A23L 2/39; A61K 36/9068; A61K 36/54

(52) U.S. Cl. CPC A61K 39/36; A61K 36/9068; A61K 36/258; A61K 31/375; A61K 31/59; A61K 36/54; A61K 9/0095; A61K 35/644; A23L 1/08; A23L 1/302; A23L 1/303; A23L 2/02; A23L 1/3002; A23L 1/30; A23L 2/39; A23L 2/52; A61K 2039/54

(57) ABSTRACT

A composition for preventing and/or alleviating an allergy related condition and methods of making such compositions are disclosed herein. A composition in accordance with a particular embodiment can include honey having pollen derived from a known plant species and/or from a known geographic location. The composition can also include a herbal and/or vitamin supplement, wherein the supplement has an anti-inflammatory property. The composition can be in liquid form, such as suitable for drinking, or in a powdered form suitable for reconstitution in liquid.

### CROSS-REFERENCE TO RELATED APPLICATION(S)

The present application claims priority to U.S. Provisional Patent Application No. 61/766,651, filed Feb. 19, 2013, which is incorporated by reference herein in its entirety.

### TECHNICAL FIELD

The present technology relates generally to beverage compositions, formulations and compositions for alleviation or prevention of allergy symptoms and related conditions, and methods for making such beverages. In particular, several embodiments are directed to beverage compositions for alleviation of patients suffering from seasonal or environmental-induced allergy symptoms and allergic conditions.

### BACKGROUND

Environment or seasonal allergies and associated symptoms occur when a patient's immune system is hypersensitive to typically harmless substances (e.g., allergens) found in the environment. These acquired allergic reactions and associated inflammatory response are a result of activation of mast cells, basophils and eosinophils by immunoglobulin E antibodies in the body. Environmental or seasonal allergies, like hay fever, can be common and can cause symptoms of varying severity, such as red and itchy/watery eyes, runny nose, itching of mucosal membranes, eczema (atopic dermatitis), hives, or asthma. Treatments for allergies typically include avoiding known allergens, use of medications such as anti-histamines, topical or oral steroids or leukotriene antagonists which are aimed at alleviating symptoms. Many of these are over-the-counter or prescription medications that are administered orally, through inhalers or topically applied. Additional therapies can include long-term regimens of allergy shots (e.g., immunotherapy) designed to produce an immune response in a patient to effectively “immunize” the patient to the allergen. While many of these drugs can reduce symptoms, they can have limited efficacy, compliance issues and they are typically associated with side effects such as dry nasal cavities, drowsiness and other undesirable conditions such as an elevated or irregular heart rate.

### DETAILED DESCRIPTION

The present disclosure is generally directed to formulations and compositions, such as beverage compositions, for alleviation or prevention of seasonal or environmental-induced allergy symptoms and methods of making the same. More particularly, the present disclosure describes beverage compositions and other formulations that can be ingested by consumers to alleviate or prevent allergy-related symptoms caused by histamine and inflammatory responses in the consumer. The following description provides specific details for a thorough understanding of and enabling description for, embodiments of the technology. However, one skilled in the art will understand that the technology may be practiced without these details. In other instances, well-known components, derivatives, substitutes and functions have not been shown or described in detail to avoid unnecessarily obscuring the description of the embodiments of the disclosure.

In one embodiment of the technology, for example, a beverage composition can be a liquid, water-based solution that includes one or more health promoting components such as nutrients or vitamins associated with reducing histamine and/or inflammatory responses in the body. For example, in one embodiment, the composition can include one or more of vitamin C and vitamin B (e.g., B₁ or thiamine, B₂ or riboflavin, B₃ or niacin or niacinainide or vitamin PP. B₅ or pantothenic acid, B₆ pyridoxine hydrochloride or pyridoxatnine, B₇ or biotin, B₉ or folic acid, B₁₂). Additional oil-soluble vitamins such as vitamin A, vitamin D (e.g., vitamin D2, vitamin D3), and vitamin E may also be incorporated. In some embodiments, such vitamins can be incorporated with a suitable dispersing agent.

In some embodiments, the composition can include herbal components such as ginger root extracts (e.g., zingiber extract) which can have anti-cancer and anti-inflammatory effects. In other embodiments, the composition can include ginseng (e.g., Panax ginseng) which may have a variety of therapeutic effects such as acting as an adaptogen homeostasis or metabolic regulator) or may prevent or decrease respiratory distress. Additional antioxidant, homeostasis regulating, anti-inflammatory and flavoring and/or coloring components may also be present in the beverage compositions. For example, the composition may include cinnamon and/or fruit juices (e.g., pomegranate, blueberry).

The beverage composition of the present disclosure can also include honey. Honey bees are used to both cross-pollinate flowers and other vegetation as well as to produce honey from both the nectar and pollen and/or spores from the vegetation visited by the honey bees. By way of theory, the pollen and/or spores incorporated into the honey can be consumed in the beverage composition or formulation and used to trigger an immune system response to produce antibodies against the introduced pollen and spores. In one embodiment, for example, consuming the honey-containing composition can introduce pollen and/or spores from flowers, grasses and pollinating trees to the consumer in small amounts such that the consumer's body will become more accustomed to the presence of the pollen and/or spores and will have a decreased histamine response when exposed to (e.g., through physical contact or inhaled) the allergens in the environment, Since the concentration of pollen and/or spores found in honey is relatively low, for example compared to directly inhaling pollen from a flower, the production of antibodies should not trigger symptoms similar to an allergic reaction. In many instances, the composition consumer will not experience any histamine or allergy-related reaction when consuming the beverage or other composition,

In one embodiment, the beverage composition or formulation can be region or geographically designed and distributed. For example, “local” honey, which is produced by honey bees pollinating and producing honey from specific species of flowers or grasses can be used to produce region-specific beverage compositions. For example, if the consumer lives in Austin, Texas, the consumer can ingest a composition that includes honey or honey extract produced by honey bees in the Austin, Texas area. Accordingly, the consumer will be exposed and may develop antibodies to pollens that the consumer would be exposed to white living in that geographical region, In other embodiments, the composition may include honey produced from honey bees pollinating/extracting pollen and nectar from known plant species. For example, the honey source can be selected based on a presence of pollen from a specific species of flower, tree, shrub and/or grass. For example, if a consumer lives in a region with a high population of cedar trees, the consumer can ingest a composition that includes honey or honey extract produced by honey bees located in a region having a high cedar tree population. Accordingly, the composition can be suitable for region-specific distribution and/or consumption or, in other embodiments, for allergen-specific distribution and/or consumption.

Methods of making a composition for the prevention and/or assisting in the reduction of allergy related symptoms in accordance with certain aspects of the technology can include providing honey that includes pollen and/or nectar derived from a known plant species, or in some embodiments, from a known geographic region. In a particular example, methods can include assessing a known or suspected plant-based allergen source for a particular cohort of human subjects that are experiencing or have a history of experiencing allergy related conditions associated with the allergen source, In such an example, providing the honey for the composition can include providing honey that includes pollen from the known or suspected plant-based allergen source such that the composition can be configured to prevent and/or alleviate allergy related conditions in the particular cohort of human subjects.

As described above, the composition can be in liquid form and consumed as a beverage. In another embodiment, the composition can be dehydrated or formed in a dry composition (e.g., powder) that can be reconstituted in liquid form or taken in a capsule or pill formulation. For example, a mixture of honey and a herbal and/or vitamin supplement can be dissolved in water to form a drinkable composition. In other embodiments, the mixture can be heated for a period of time sufficient to reduce a water content of the mixture and cooled to produce a dried composition. In one embodiment, the dried composition can be a chewable or dissolvable tablet, Additionally, the concentration of the components of the composition can be formulated to suit desired taste, texture and consistency of the beverage or dry formulation.

### EXAMPLES

The disclosure is further illustrated, but is not intended to be limited, by the following examples.

### Example 1

### Beverage Formulation

The following components can be combined:

250 mg-1 gram Zingiber officinale

0-250 mg Panax ginseng

50-250 mg cinnamon

100-500 mg vitamin C

400-1000 IU vitamin D

1-5 mL Honey

The above ingredients can be dissolved in water. Variations can include the addition of fruit juice, such as pomegranate juice, blueberry juice, etc. Additionally, the components can be dehydrated or in powdered form for reconstitution before drinking. In another arrangement, the components can be in dry form for incorporation into capsule or pill (e.g., tablet formulation) for oral administration, Further, in some embodiments, one or more of the foregoing ingredients may not included.

### CONCLUSION

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while process steps, formulation components or functions are presented in a given order, alternative embodiments may include these in a different order, or substantially concurrently. The teachings of the disclosure provided herein can be applied to other compositions, not only the compositions described herein. The various embodiments described herein can be combined to provide further embodiments.

Specific elements of any of the foregoing embodiments can he combined or substituted for elements in other embodiments. Furthermore, while aspects associated with certain embodiments of the disclosure have been described in the context of these embodiments, other embodiments may also exhibit such aspects, and not all embodiments need necessarily exhibit such aspects to fall within the scope of the disclosure. Accordingly, the disclosure is not limited, except as by the appended claims.

1. A composition for therapeutically treating an allergy related condition in a human patient, the composition comprising:
honey including pollen from a known geographic location, wherein the pollen is associated with the allergy related condition of the patient; and
a herbal or vitamin supplement, wherein the supplement has an anti-inflammatory property,
wherein the composition comprises 1-5 mL of honey and 50 mg-1 g of supplement wherein the honey and supplement are provided in a ratio that prevents or alleviates the allergy related condition in the patient.

2. The composition of claim 1 wherein the composition is dissolved in water.

3. The composition of claim 1 wherein the composition further includes fruit juice.

4. The composition of claim 1 wherein the supplement includes a ginger root extract.

5. The composition of claim 1 wherein the supplement includes ginseng.

6. The composition of claim 1, further comprising 100-500 mg of vitamin C and 400-1000 IU of vitamin D.

7. The composition of claim 1, further comprising 50-250 mg of cinnamon.

8. The composition of claim 1 wherein the pollen is from a known plant.

9. The composition of claim 1 wherein the composition is a powdered composition.

10. The composition of claim 1 wherein the composition is in a capsule or tablet formulation.

11. A beverage composition for therapeutically treating an allergic condition of a human patient, the composition comprising:
honey including pollen derived from a known plant species, wherein the known plant species is a flower, tree, shrub, or grass known to produce pollen that causes allergy-related symptoms in the patient; and
an herbal supplement having an anti-inflammatory property,
wherein the composition includes 1-5 mL of honey and 50 mg-1 g of the herbal supplement,
wherein the honey and herbal supplement are provided in a predetermined ratio that inhibits or prevents allergy-related histamine and inflammatory responses of the patient.

12. (canceled)

13. A method of making a composition for preventing or reducing an allergy related condition in a human patient, the method comprising:
providing honey including pollen derived from a known plant species and/or a known geographical location; and
adding a herbal and/or vitamin supplement to the honey to form a mixture, wherein the supplement has an anti-inflammatory property.

14. The method of claim 13, further comprising assessing a known or suspected plant-based allergen source for a cohort of human subjects prior to providing the honey, wherein providing the honey includes providing honey that includes pollen from the known or suspected plant-based allergen source.

15. The method of claim 13, further comprising heating the mixture for a period of time sufficient to reduce a water content of the mixture, and cooling the mixture to produce a dried composition.

16. The method of claim 13, further comprising dissolving the mixture in water to form a drinkable composition.
